(19) United States
(12) Patent Application Publication
(10) Pub. No.: US 2003/0118572 A1
(43) Pub. Date: Jun. 26, 2003

(54) Modified enzymes

(75) Inventors: Heinz Josef Deussen, Soborg (DK); Claus Crone Fuglsang, Niva (DK); Arne Agerlin Olsen, Virum (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/237,451

(22) Filed: Sep. 9, 2002

Related U.S. Application Data
(63) Continuation of application No. 09/217,865, filed on Dec. 21, 1998, now abandoned.
(63) Continuation of application No. 09/198,956, filed on Nov. 24, 1998, now Pat. No. 6,165,769.
(60) Provisional application No. 60/070,751, filed on Jan. 8, 1998.

(30) Foreign Application Priority Data
Dec. 29, 1997 (DK) 1547/97

(51) Int. Cl. A61K 38/43; C12N 9/00

(52) U.S. Cl. 424/94.1; 435/183

(57) ABSTRACT

The present invention relates to modified enzymes comprising an enzyme and at least one polyanionic domain, e.g. polyglutamic acid, polyaspartic acid or a polycarboxylic acid, wherein the enzyme comprises or is covalently attached to each said polyanionic domain. The present invention also relates to oral care compositions comprising such modified enzymes and use of the oral care compositions for preventing or treating dental disease, in particular for preventing or removing plaque. The modified enzymes are able to bind to hydroxylapatite in teeth.

### CROSS-REFERENCE TO RELATED APPLICATIONS

This application is a continuation of U.S. application Ser. No. 09/198,956 filed Dec. 21, 1998, and claims, under 35 U.S.C. 119, the benefit of U.S. provisional application No. 60/070,751 filed Jan. 8, 1998, and priority of Danish application no. PA 1997 01547 filed Dec. 29, 1997, the contents of which are fully incorporated herein by reference.

### BACKGROUND OF THE INVENTION

1. Field of the Invention

The present invention relates to modified enzymes comprising a polyanionic domain, to methods for producing such modified enzymes, to oral compositions comprising such modified enzymes, and to the use of such oral care compositions for the prevention and/or removal of dental plaque.

2. Description of the Related Art

Dental plaque is a mixture of bacteria, epithelial cells, leukocytes, macrophages and other oral exudate that is formed on the surface of teeth. The formation of dental plaque leads to dental caries, gingival inflammation, periodontal disease, and eventually tooth loss. Said bacteria produce highly branched polysaccharides, which together with micro-organisms from the oral cavity form an adhesive matrix for the continued proliferation of plaque.

As plaque continues to accumulate, rock hard white or yellowish deposits arise. These deposits are called calcified plaque, calculus or tartar, and are formed in the saliva from plaque and minerals, in particular calcium.

Oral Polysaccharides

Oral polysaccharides are produced from sucrose introduced into the mouth, e.g. as a food or beverage constituent, by the action of cariogenic micro-organisms such as Streptococcus mutans or Streptococcus sanguis growing in the oral cavity.

Said oral polysaccharides comprise water-soluble dextran having large portions of alpha-1,6-glycosidic linkages, and a major component of water-insoluble extra-cellular polysaccharides called “mutan” comprised of a backbone with alpha-1,3-glycosidic linkages and branches with alpha-1,6-glycosidic linkages.

Mutan binds to hydroxylapatite (constituting the hard outer porous layer of the teeth) and to acceptor proteins on the cell surface of said cariogenic bacteria adhering to the tooth surface.

To prevent the formation of dental caries, plaque, and tartar, it has been suggested to add various enzymes, e.g. a dextranase and/or a mutanase, to oral care compositions and products, and a number of oral care products containing various enzymes, including glucanases, oxidoreductases such as oxidases and peroxidases, are known.

A problem with the known enzyme-containing oral care products, however, is the fact that the enzymes generally do not bind to components of the teeth or plaque, which means that enzymes applied e.g. by means of a toothpaste are relatively quickly removed from the teeth and mouth. This in turn means that such enzymes are able to act only for a limited amount of time, and that their full potential for the maintenance of oral hygiene by e.g. combating plaque is not realised.

Chu and orgel (Bioconjugate Chem., 8, 103 (1997) found that the decamer of Glutamic acid and the trimer of phosphonated valeric acid can be conjugated to biotin, and that the conjugates can be used to mediate the binding of the biotin-binding protein avidin to hydroxylapatite. The article suggests that anionic peptides might be used as carriers of ligands to bone.

Hosain et al. (J. Nucl. Med., 37:105-107, Jan. 1996) reported that a methotrexate-biphosphonate conjugate containing a peptide bond behaved like a bone-seeking agent. The authors suggest, based on this finding, the possibility for specific delivery of antineoplastic agents to bone tumor sites.

It has now surprisingly been found that a modified enzyme comprising one or more polyanionic domains binds to hydroxylapatite in the teeth, thereby allowing the enzyme in an oral care composition to exert a prolonged enzymatic action.

### SUMMARY OF THE INVENTION

It is thus an object of the present invention to provide a modified enzyme that comprises or are coupled to polyanionic domains, as well as oral compositions comprising such modified enzymes.

In a first aspect, the present invention thus relates to a modified enzyme comprising an enzyme and at least one polyanionic domain, wherein the enzyme comprises or is covalently attached to each said polyanionic domain.

A second aspect the invention relates to an oral care composition comprising such modified enzymes.

A third aspect the invention relates to the use of a composition or oral care product comprising the modified enzymes of the invention for the prevention or treatment of a dental disease, in particular for preventing the formation of dental plaque or removing dental plaque.

### DETAILED DESCRIPTION OF THE INVENTION

As used herein, the term “modified enzyme” refers to an enzyme comprising or covalently attached to at least one polyanionic domain. The attachment may be effected by coupling a polyanionic domain to various groups in the enzyme by chemical or recombinant DNA techniques, or the polyanionic domain may be inserted into one or more sites of the enzyme by means of recombinant DNA technology.

In a preferred embodiment of the invention, at least one polyanionic domain is covalently attached to a carboxylate group and/or an amino group of the enzyme.

In a further preferred embodiment of the invention, the enzyme moiety is chemically modified by coupling a polyanionic domain to the carboxyl group of Glutamic acid and/or Aspartic acid residues in the enzyme and/or to one or more C-terminal carboxyl groups in the enzyme.

In a still further preferred embodiment of the invention, the modified enzyme is produced by means of recombinant DNA technology, i.e. the polyanionic domain constitutes an extension of the enzyme in question by being bound to one or more C- and/or N-terminal groups in the enzyme, or the polyanionic domain is incorporated into one or more sites in the enzyme.

In the present context the term “polyanionic domain” is intended to mean a molecule or moiety having a net negative charge at pH 7 and being capable of being covalently bound to an enzyme. Alternatively, the polyanionic domain may be incorporated into the amino acid sequence of the enzyme itself. Suitable domains which may be used according to the invention are peptides comprising from 1 to 150 amino acid residues, such as from 1 to 100, e.g. from 1 to 50, preferably from 2 to 40, such as from 2 to 30, e.g. from 2 to 20, more preferably from 3 to 15, such as from 3 to 10. Any naturally-occurring amino acid may be incorporated in the domains' peptide structure. It is contemplated that also D-enantiomers of naturally-occurring amino acids, as well as beta-amino acids may be comprised in the domain. When the domain is a peptide, it is of course a requirement that the peptide domain possesses a net negative charge at pH 7. Thus, when the peptide domain does not comprise any positively charged amino acid residues, the peptide domain must include at least one glutamic acid and/or aspartic acid residue, e.g. from 1 to 150, such as from 1 to 100, e.g. from 1 to 50, preferably from 2 to 40, such as from 2 to 30, e.g. from 2 to 20, more preferably from 3 to 15, such as from 3 to 10.

Preferred examples of polyanionic peptide domains are polyglutamic acid and polyaspartic acid comprising a total of from 2 to 100 glutamic acid and/or aspartic acid residues, such as from 3 to 75, e.g. from 3 to 50, preferably from 3 to 40, such as from 3 to 30, e.g. from 3 to 20, more preferably from 3 to 15, such as from 3 to 10, e.g. from 4 to 8.

It is contemplated that polyanionic peptides containing polyglutamic acid and/or polyaspartic acid together with at least one amino acid with an uncharged side chain will also be efficient domains. Thus, such amino acids with an uncharged side chain may be incorporated in the polyanionic peptide in several ways. In some cases it might be advantageous to separate the individual negative charges, generated by the Asp and/or Glu side chains, by alternate insertion of Glu and/or Asp residues and amino acids with an uncharged side chain such as alanine, valine, leucine, isoleucine, methionine, phenylalanine, tryptophan, proline, glycine, serine, threonine, cysteine, tyrosine, aspargine, and/or glutamine.

It is also contemplated that in some cases it might be advantageous that various domains in the polyanionic peptide possess a negative charge, whereas other domains remain uncharged, i.e. when amino acids with an uncharged side chain are incorporated in the polyanionic peptide domain, said amino acids with uncharged side chains may optionally be located together in one or more groups of 2 to 50 residues, preferably 3 to 25 residues, such as 3 to 10 residues, e.g. 4 to 8 residues.

Specific examples of suitable polyglutamic acids and polyaspartic acids are Glu-Glu, (Glu)₃, (Glu)₄, (Glu)₅, (Glu)₆, (Glu)₇, (Glu)₈, (Glu)₉, (Glu)₁₀, Asp-Asp, (Asp)₃, (Asp)₄, (Asp)₅, (Asp)6, (Asp)₇, (Asp)₈, (Asp)₉, (Asp)₁₀, Glu-Asp, (Glu-Asp)₂, (Glu-Asp)₃, (Glu-Asp)₄, (Glu-sp)₅, Asp-Glu, (Asp-Glu)₂, (Asp-Glu)₃, (Asp-Glu)₄, (Asp-Glu)₅, Xaa-Glu, (Xaa-Glu)₂, (Xaa-Glu)₃, (Xaa-Glu)₄, (Xaa-Glu)₅, (Xaa-Glu)₆, (Xaa-Glu)₇, (Xaa-Glu)₈, (Xaa-Glu)₉, (Xaa-Glu)₁₀, Glu-Xaa, (Glu-Xaa)₂, (Glu-Xaa)₃, (Glu-Xaa)₄, (Glu-Xaa)₅, (Glu-Xaa)₆, (Glu-Xaa)₇, (Glu-Xaa)₈, (Glu-Xaa)₉, (Glu-Xaa)₁₀, Xaa-Asp, (Xaa-Asp)₂, (Xaa-Asp)₃, (Xaa-Asp)₄, (Xaa-Asp)₅, (Xaa-Asp)₆, (Xaa-Asp)₇, (Xaa-Asp)₈, (Xaa-Asp)₉, (Xaa-Asp)₁₀, Asp-Xaa, (Asp-Xaa)₂, (Asp-Xaa)₃, (Asp-Xaa)₄, (Asp-Xaa)₅, (Asp-Xaa)₆, (Asp-Xaa)₇, (Asp-Xaa)₈, (Asp-Xaa)₉, (Asp-Xaa)₁₀, Xaaₐ-(Glu or Asp)_{b}-Xaa_{c}-(Glu or Asp)_{d}-Xaaₑ, wherein Xaa denotes an amino acid with an uncharged side chain, such as alanine, valine, leucine, isoleucine, methionine, phenylalanine, tryptophan, proline, glycine, serine, threonine, cysteine, tyrosine, aspargine or glutamine, and a, b, c, d and e are integers in the range of from 0 to 25.

Other interesting polyanionic domains which can be used according to the invention are phosphono derivatives of Formula I

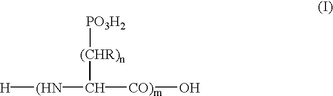

wherein n is an integer in the range of from 1 to 15, preferably in the range of from 1 to 8, such as from 1 to 5, e.g. from 1 to 3, m is an integer in the range of from 1 to 50, preferably from 2 to 40, such as from 2 to 30, e.g. from 2 to 20, more preferably from 3 to 15, such as from 3 to 10, and each R is independently selected from the group consisting of hydrogen, C₁₋₆-alkyl, C₁₋₆-alkenyl, hydroxy, amino, and halogen such as fluoro, chloro, iodo and bromo. Preferably, R is hydrogen.

Specific examples of suitable polyanionic domains of the general formula I are trimers (m=3), tetramers (m=4), pentamers (m=5), hexamers (m=6), pentamers (m=7), octamers (m=8), nonamers (m=9), decamers (m=10), and mixtures thereof, of 2-amino-3-phosphono propionic acid (n=1, R=H), 2-amino-4-phosphono butyric acid (n=2, R=H), 2-amino-5-phosphono valeric acid (n=3, R=H), and/or 2-amino-6-phosphono caproic acid (n=4, R=H).

In the present context, the term “C₁₋₆-alkyl” used alone or as part of another group designates a straight, branched or cyclic saturated hydrocarbon group having from one to six carbon atoms such as methyl, ethyl, n-propyl, iso-propyl, n-butyl, iso-butyl, sec-butyl, tert-butyl, n-pentyl, n-hexyl, cyclohehexyl, etc.

In a similar way, the term “C₂₋₆-alkenyl” designates a hydrocarbon group having from two to six carbon atoms, which may be straight, branched or cyclic and may contain one or more double bonds such as vinyl, allyl, 1-butenyl, 2-butenyl, iso-butenyl, 1-pentenyl, 2-pentenyl, 4-pentenyl, 3-methyl-1-butenyl, 2-hexenyl, 5-hexenyl, cyclohexenyl, 2,3-dimethyl-2-butenyl, etc., and which may have the cis and/or trans configuration.

Still other polyanionic domains which are envisaged to be suitable for the purpose of the invention are polyphosphates, polysulfonic acids, and polycarboxylic acids.

The term “polyphosphate” is intended to mean a molecule comprising at least two and preferably at least three phosphate groups. If a phosphate group of such a polyphosphate is used for coupling to an amine group in the polypeptide, the polyphosphate should then preferably contain at least 3 phosphate groups. Preferred polyphosphates are aminated polyphosphates.

The term “polysulfonic acid” is intended to mean a molecule comprising at least two and preferably at least three sulfonic acid groups. If a sulfonic acid group of such a polysulfonic acid is used for coupling to an amine group in the polypeptide, the polysulfonic acid should then preferably contain at least 3 sulfonic acid groups. Preferred polysulfonic acids are aminated polysulfonic acids.

The term “polycarboxylic acid” is intended to mean a molecule comprising at least two and preferably at least three carboxyl groups. If a carboxyl group of such a polycarboxylic acid is used for coupling to an amine group in the polypeptide, the polycarboxylic acid should then contain at least 3 carboxyl groups. An example of a suitable polycarboxylic acid is citric acid.

A preferred class of polycarboxylic acid is an aminated polycarboxylic acid. Examples of aminated polycarboxylic acids are aminated polycarboxylic alkanes and derivatives thereof, aminated polycarboxylic sugars, aminated polycarboxylic alcohols and aminated polycarboxylic polyalcohols. Specific examples of suitable aminated polycarboxylic acids are aminated poly(vinyl acetate-co-crotonic acid), aminated polygalacturonic acid, and aminated poly(acrylamide-co-acylic acid).

In general, the aminated polycarboxylic acids, such as aminated polycarboxylic alkanes, aminated polycarboxylic sugars, aminated polycarboxylic alcohols and aminated polycarboxylic polyalcohols, should have at least one amino group per molecule, but they may suitably also have more than one amino group per molecule.

The polyanionic domain may be covalently coupled to the enzyme by various methods which, of course, will depend on the actual chosen attachment group or groups in the enzyme and the polyanionic domain, respectively. Thus, for the person skilled in the art, a broad class of chemical coupling techniques are available. However, preferred methods for chemically coupling the polyanionic domain to the enzyme are e.g. those described in G. T Hermanson “Bioconjugate Techniques”, Academic Press, 1996, and G. T. Hermanson et al. “Immobilized Affinity Ligand Techniques”, Academic Press, 1992.

The general strategy for coupling a polyanionic domain to an enzyme usually comprises reacting one or more functional groups in the enzyme with one or more functional groups in the polyanionic domain, optionally with the aid of suitable catalysts or other coupling promoting agents. Another strategy commonly applied in coupling procedures involves the transformation of functional groups in the enzyme and/or the polyanionic domain into reactive groups and subsequently coupling the reactants, i.e. the enzyme and the polyanionic domain.

Examples of suitable coupling reaction techniques which can be employed for the production of the modified enzymes are e.g. reaction techniques using amine groups, thiol groups, carboxylate groups, hydroxyl groups, aldehyde/ketone groups, active hydrogen groups and photo-reactive groups.

As amine groups are capable of reacting with e.g. isothiocyanates, isocyanates, acyl azides, NHS-esters, sulfonyl chlorides, aldehydes, glyoxals, epoxides, oxiranes, carbonates, arylating agents, imidoesters, anhydrides, acid groups activated with carbodiimides, and photoreactive groups such as aryl azides, benzophenones, diazo compounds and diaziridine derivatives, the formation of such groups in the enzyme or the polyanionic domain may be used to covalently couple the polyanionic domain to the enzyme.

In a similar way, thiol-reactive groups such as e.g. haloacetyls, alkyl halide derivatives, maleimides, aziridines, acryloyl derivatives, arylating agents, thiol-disulfide exchange reagents such as pyridyl disulfides, TNB-thiol, and disulfide reductants may conveniently be used for the formation of covalent bonds between the polyanionic domain and the enzyme, through thiol groups in the enzyme or the polyanionic domain.

Other suitable coupling strategies include the use of carboxylate-reactive groups such as diazoalkanes, diazoacetyl compounds, CDI and carbodiimides; hydroxyl-reactive groups such as epoxides, oxiranes, CDI, N,N′-disuccinimidylcarbonate, N-hydroxysuccinimidyl chloroformate, alkyl halogens, isocyanates, and formation of reactive aldehyde groups from the hydroxyl groups by means of periodate oxidation or enzymatic oxidation; aldehyde/ketone reactive groups such as hydrazine and reactions such as Schiff-base formation, reductive amination, and Mannich condensation; active hydrogen-reactive groups such as diazonium derivatives and iodination reactions.

Preferably, the polyanionic domain is covalently bound to the enzyme by means of a C—N bond, the carbon atom preferably originating from the enzyme and the nitrogen atom preferably originating the polyanionic domain. In a preferred embodiment, wherein the polyanionic domain is a peptide having an overall negative charge at pH 7.0, the covalent bond is a peptide bond, wherein the carbon atom preferably originates from the enzyme and the nitrogen atom preferably originates the peptide.

Methods and coupling agents for establishing C—N bonds, including peptide bonds, are well-known in the art, see e.g. J. Jones “The Chemical Synthesis of Peptides”, Clarendon Press, Oxford, 1991, and M. Bodanszky and A. Bodanszky “The Practice of Peptide Synthesis”, Springer-Verlag, Berlin, 1994.

A particularly preferred coupling agent for the coupling reaction is a carbodiimide, e.g. 1-ethyl-3-(3-dimethylaminopropyl)carbodiimide (EDC).

Methods of conjugating proteins with domains using EDC can be implemented according to manufacturers' descriptions (e.g. Pierce Instructions 0475 C, 22980 X; 22981 X; EDC) using either the protocol for “Use of EDC for coupling of Haptens/small ligands to carrier Proteins” or “Protocol for Efficient Two-Step coupling of Proteins in Solution Using EDC and N-hydroxysuccinimide or sulfo-N-hydroxysucciminide”.

Furthermore, if the polyanionic domain contains free amine groups, such groups may conveniently be protected by methods well-known in the art, see e.g. J. Jones “The Chemical Synthesis of Peptides”, Clarendon Press, Oxford, 1991, and M. Bodanszky and A. Bodanszky “The Practice of Peptide Synthesis”, Springer-Verlag, Berlin, 1994. Thus, amine groups of the polyanionic domain, such as a peptide, may be protected by e.g. tert-butyloxycarbonyl (BOC) before activation with EDC takes place. After having completed the conjugation, the protecting groups may be removed using standard techniques, such as removing the BOC protecting group with e.g. trifluoro acetic acid.

For example, the enzyme may be dissolved, or transferred by dialysis or desalting by size exclusion chromatography in a coupling buffer, for example 50 mM MES pH 5.0 containing 200 mM sodium chloride. The polyanionic domain, e.g. a peptide and/or a polycarboxylic acid, may be dissolved in the coupling buffer as well. The conjugation reaction may proceed by mixing enzyme and domain to a final concentration of e.g. 3 mg/ml for both enzyme and domain, followed by mixing with e.g. 5 mg of EDC per mg of enzyme. The conjugation reaction then runs for e.g. about 2 hours at room temperature with continuous stirring. The reaction is terminated by removal of surplus reagent either by desalting by size exclusion chromatography or by extensive dialysis, e.g. against 0.2 M ammonium acetate pH 6.9 at 5° C. The resulting derivative may then be stored at 5° C.

In a preferred method, the enzyme is first activated by EDC in the “Two-Step Coupling of Proteins” method, followed by removal of excess EDC by dialysis or desalting. The conjugation reaction may proceed by mixing activated enzyme and the domain, e.g. peptide and/or polycarboxylic acid, and the derivative can be subsequently purified using standard procedures.

The degree of modification or incorporation of domains may, of course, be controlled by adjustments in the initial enzyme, domain and/or carbodiimide concentration. Variations in pH or temperature of the coupling buffer may also be used to optimise the conjugation reaction for a specific enzyme.

Active site protection by substrate, substrate analogues or reversible inhibitors may be used to control the modification reaction.

In another preferred embodiment of the invention, the enzyme may be modified through attachment of the above-mentioned domains to the carbohydrate part of glycosylated enzymes.

Periodate oxidation of carbohydrates is a well-established classical technology for generation of aldehyde groups which readily react with amino groups on the polyanionic domain, initially generating a Schiff base. The reaction product can be stabilised by standard methods, e.g. by reduction using NaBH₄ or NaCNBH₃ (see e.g. G. T Hermanson, Bioconjugate Techniques, Academic Press, 1996). This process may be performed as a one-step or two-step procedure, and a number of parameters may be varied to optimise the reaction conditions for a specific enzyme/or a specific application.

In another preferred embodiment of the invention, the enzyme may be modified by substitution and/or addition of one or more amino acids by means of recombinant DNA-technology. The invention therefore further relates to a modified enzyme comprising a modified enzyme. The enzyme modification may e.g. be:

i) insertion of at least one glutamic acid and/or aspartic acid residues in one or more sites of the enzyme, such as insertion of e.g. from 1 to 10 glutamic acid and/or aspartic acid residues, preferably from 1 to 7 glutamic acid and/or aspartic acid residues, e.g. from 1 to 5 glutamic acid and/or aspartic acid residues;

ii) insertion of at least one loop comprising at least one glutamic acid or aspartic acid residue, such as EEEEEEEEEEEEEEEEE, DDDDDDDDDDDDDDDDD, DEDEDEDEDEDEDEDED EPEPEPEPEPEPEPEPE, DPDPDPDPDPDPDPDPD, DADADADADADADADAD, the length of the above-mentioned sequences as well as the number of aspartic acid residues, glutamic acid residues, and amino acids with an uncharged side chain may, of course, vary within a broad range depending on the enzyme in question and the desired properties of the modified enzyme;

iii) extension of one or more N- and/or C-terminal in the enzyme. Preferred examples of amino acid sequences which can constitute the extension may be such as described earlier, e.g. polyglutamic acid and polyaspartic acid comprising a total of from 2 to 100 glutamic acid and/or aspartic acid residues, such as from 3 to 75, e.g. from 3 to 50, preferably from 3 to 40, such as from 3 to 30, e.g. from 3 to 20, more preferably from 3 to 15, such as from 3 to 10, e.g. from 4 to 8.

The above-mentioned insertions and N- and C-terminal extensions may conveniently be carried out by means of recombinant DNA-technology using general methods and principles known to the person skilled in the art.

Oral Care Compositions

Although the oral care compositions or products of the invention have as a primary function the prevention and/or removal of dental plaque by the enzymatic action of modified enzymes bound to hydroxylapatite of the teeth, such compositions or products may also directly or indirectly have other oral care functions at the same time, e.g. the prevention of dental cavities, gingivitis and periodontal disease in general.

The enzyme moiety of the modified enzymes according to the invention may be any enzyme suitable for the desired purpose. It is in particular an enzyme selected from the group consisting of oxidoreductases such as oxidases and peroxidases, proteases, lipases, glucanases, esterases, deaminases, ureases and polysaccharide hydrolases, or a mixture thereof.

Preferred enzyme activities for oral care compositions are glucanases activities, such as an alpha-glucosidase activity, such as dextranase, mutanase, and/or pullulanase activity.

Relevant glucanases include the enzymes in the enzyme class EC 3.2.1, in particular:

glucan 1,4-alpha-glucosidase (3.2.1.3), cellulase (3.2.1.4), endo-1,3(4)-beta-glucanase (3.2.1.6), endo-1,4-beta-xylanase (3.2.1.8), dextranase (3.2.1.11), chitinase (3.2.1.14), polygalacturonase (3.2.1.15), lysozyme (3.2.1.17), beta-glucosidase (3.2.1.21), alpha-galactosidase (3.2.1.22), beta-galactosidase (3.2.1.23), amylo-1,6-glucosidase (3.2.1.33), xylan 1,4-beta-xylosidase (3.2.1.37), glucan endo-1,3-beta-D-glucosidase (3.2.1.39), alpha-dextrin endo-1,6-glucosidase (3.2.1.41), sucrose alpha-glucosidase (3.2.1.48), glucan endo-1,3-alpha-glucosidase (3.2.1.59), glucan 1,4-beta-glucosidase (3.2.1.74), glucan endo-1,6-beta-glucosidase (3.2.1.75), arabinan endo-1,5-alpha-arabinosidase (3.2.1.99), lactase (3.2.1.108) and chitonanase (3.2.1.132).

Examples of relevant glucanases include alpha-1,3-glucanases derived from Trichoderma harzianum; alpha-1,6-glucanases derived from Paecilomyces; beta-glucanases derived from Bacillus subtilis; beta-glucanases derived from Humicola insolens; beta-glucanases derived from Aspergillus niger; beta-glucanases derived from a strain of Trichoderma; beta-glucanases derived from Oerskovia xanthineolytica; exo-1,4-alpha-D-glucosidases (glucoamylases) derived from Aspergillus niger.

Contemplated are also microbial amylases such as alpha-amylases derived from Bacillus subtilis; alpha-amylases derived from Bacillus amyloliquefaciens; alpha-amylases derived from Bacillus stearothermophilus; alpha-amylases derived from Aspergillus oryzae; alpha-amylases derived from non-pathogenic micro-organisms.

Further, contemplated suitable glucanases include alpha-galactosidases derived from Aspergillus niger; Pentosanases, xylanases, cellobiases, cellulases, hemi-cellulases derived from Humicola insolens; cellulases derived from Trichoderma reesei; cellulases derived from non-pathogenic mold; pectinases, cellulases, arabinases, hemi-celluloses derived from Aspergillus niger; dextranases derived from Penicillium lilacinum; endo-glucanase derived from non-pathogenic mold; pullulanases derived from Bacillus acidopullyticus; beta-galactosidases derived from Kluyveromyces fragilis; xylanases derived from Trichoderma reesei.

Specific examples of readily available commercial glucanases include Alpha-Gal®, Bio-Feed® Alpha, Bio-Feed® Beta, Bio-Feed® Plus, Novozyme® 188, Carezyme®, Celluclast®, Cellusoft®, Ceremyl®, Citrozym®, Denimax®, Dezyme®, Dextrozyme®, Finizym®, Fungamyl®, Gamanase®, Glucanex®, Lactozym®, Maltogenase®, Pentopan®, Pectinex®, Promozyme®, Pulpzyme®, Novamyl®, Termamyl®, AMG (Amyloglucosidase Novo), Sweetzyme®, Aquazym® (all enzymes available from Novo Nordisk A/S). Other carbohydrases are available from other companies.

It is to be understood that also glucanase variants are contemplated as the enzyme moiety.

Another group of enzymes of interest are Oxidoreductases (i.e. enzymes classified under the Enzyme Classification number E.C. 1 in accordance with the Recommendations (1992) of the International Union of Biochemistry and Molecular Biology (IUBMB)).

Examples include oxidoreductases selected from those classified under the Enzyme Classification (E.C.) numbers:

Glycerol-3-phosphate dehydrogenase _NAD+_(1.1.1.8), Glycerol-3-phosphate dehydrogenase _NAD(P)⁺_(1.1.1.94), Glycerol-3-phosphate 1-dehydrogenase _NADP_(1.1.1.94), Glucose oxidase (1.1.3.4), Hexose oxidase (1.1.3.5), Catechol oxidase (1.1.3.14), Bilirubin oxidase (1.3.3.5), Alanine dehydrogenase (1.4.1.1), Glutamate dehydrogenase (1.4.1.2), Glutamate dehydrogenase _NAD(P)⁺_(1.4.1.3), Glutamate dehydrogenase _NADP+_(1.4.1.4), L-Amino acid dehydrogenase (1.4.1.5), Serine dehydrogenase (1.4.1.7), Valine dehydrogenase _NADP⁺_(1.4.1.8), Leucine dehydrogenase (1.4.1.9), Glycine dehydrogenase (1.4.1.10), L-Amino-acid oxidase (1.4.3.2.), D-Amino-acid oxidase(1.4.3.3), L-Glutamate oxidase (1.4.3.11), Protein-lysine 6-oxidase (1.4.3.13), L-lysine oxidase (1.4.3.14), L-Aspartate oxidase (1.4.3.16), D-amino-acid dehydrogenase (1.4.99.1), Protein disulfide reductase (1.6.4.4), Thioredoxin reductase (1.6.4.5), Protein disulfide reductase (glutathione) (1.8.4.2), Laccase (1.10.3.2), Catalase (1.11.1.6), Peroxidase (1.11.1.7), Lipoxygenase (1.13.11.12), Superoxide dismutase (1.15.1.1)

Glucose oxidases may be derived from Aspergillus niger.

Laccases may be derived from Polyporus pinsitus, Myceliophtora thermophila, Coprinus cinereus, Rhizoctonia solani, Rhizoctonia praticola, Scytalidium thermophilum and Rhus vernicifera.

Bilirubin oxidases may be derived from Myrothechecium verrucaria.

Peroxidases may be derived from e.g. Soy bean, Horseradish or Coprinus cinereus.

Protein disulfide reductases may be any mentioned in any of WO 95/00636, WO 95/01425 and WO 95/01420 (Novo Nordisk A/S) including Protein Disulfide reductases of bovine origin, Protein Disulfide reductases derived from Aspergillus oryzae or Aspergillus niger, and DsbA or DsbC derived from Escherichia coli.

Specific examples of readily available commercial oxidoreductases include Gluzyme™ (enzyme available from Novo Nordisk A/S). However, other oxidoreductases are available from others.

It is to be understood that also variants of oxidoreductases are contemplated as the parent enzyme.

Another group of enzymes of interest are lipases (i.e. enzymes classified under the Enzyme Classification number E.C. 3.1.1 (Carboxylic Ester Hydrolases) in accordance with the Recommendations (1992) of the International Union of Biochemistry and Molecular Biology (IUBMB)) include lipases within this group.

Examples include lipases selected from those classified under the Enzyme Classification (E.C.) numbers:

3.1.1 (i.e. so-called Carboxylic Ester Hydrolases), including (3.1.1.3) Triacylglycerol lipases, (3.1.1.4.) Phosphorlipase A₂.

Examples of lipases include lipases derived from the following microorganisms. The indicated patent publications are incorporated herein by reference:

Humicola, e.g. H. brevispora, H. lanuginosa, H. brevis var. thermoidea and H. insolens (U.S. Pat. No. 4,810,414)

Pseudomonas, e.g. Ps. fragi, Ps. stutzeri, Ps. cepacia and Ps. fluorescens (WO 89/04361), or Ps. plantarii or Ps. gladioli (U.S. Pat. No. 4,950,417 (Solvay enzymes)) or Ps. alcaligenes and Ps. pseudoalcaligenes (EP 218 272) or Ps. mendocina (WO 88/09367; U.S. Pat. No. 5,389,536).

Fusarium, e.g. F. oxysporum (EP 130,064) or F. solani pisi (WO 90/09446).

Mucor (also called Rhizomucor), e.g. M. miehei (EP 238 023).

Chromobacterium (especially C. viscosum)

Aspergillus (especially A. niger).

Candida, e.g. C. cylindracea (also called C. rugosa) or C. antarctica (WO 88/02775) or C. antarctica lipase A or B (WO 94/01541 and WO 89/02916).

Geotricum, e.g. G. candidum (Schimada et al., 1989, J. Biochem., 106, 383-388)

Penicillium, e.g. P. camembertii (Yamaguchi et al., (1991), Gene 103, 61-67).

Rhizopus, e.g. R. delemar (Hass et al., (1991), Gene 109, 107-113) or R. niveus (Kugimiya et al., (1992) Biosci. Biotech. Biochem 56, 716-719) or R. oryzae.

Bacillus, e.g. B. subtilis (Dartois et al., (1993) Biochemica et Biophysica acta 1131, 253-260) or B. stearothermophilus (JP 64/7744992) or B. pumilus (WO 91/16422).

Specific examples of readily available commercial lipases include Lipolase®, Lipolase® Ultra, Lipozyme®, Palatase®, Novozym® 435, Lecitase® (all available from Novo Nordisk A/S).

Examples of other lipases are Lumafast®, Ps. mendocian lipase from Genencor Int. Inc.; Lipomax®, Ps. Pseudoalcaligenes lipase from Gist Brocades/Genencor Int. Inc.; Fusarium solani lipase (cutinase) from Unilever; Bacillus sp. lipase from Solvay Enzymes. Other lipases are available from other companies.

It is to be understood that also lipase variants are contemplated as the suitable enzymes. Examples of such are described in e.g. WO 93/01285 and WO 95/22615.

The activity of the lipase can be determined as described in “Methods of Enzymatic Analysis”, Third Edition, 1984, Verlag Chemie, Weinhein, vol. 4, or as described in AF 95/5 GB (available on request from Novo Nordisk A/S).

Preferably, the modified enzyme of the invention has an enzymatic activity that is at least 1% of the catalytic activity of the free enzyme, preferably at least 2%, such as at least 5%, e.g. at least 10%, more preferably at least 20%, such as at least 30%, e.g. at least 40%, still more preferably at least 50%, such as at least 60%, e.g. at least 70%, even more preferably at least 80%, such as at least 90%, e.g. at least 95%, most preferably the modified enzyme is substantially identical to the catalytic activity of the free enzyme, as determined according to “Methods of Enzymatic Analysis”, 3rd. Edition, vol. 1-10, 1984, Verlag Chemie, Weinheim. Methods for determining the activity of different types classes of enzymes are found e.g. in the following volumes of this book:

| | |
|---|---|
| Oxidoreduktaser: | vol. 3 |
| Carbohydraser: | vol. 4 |
| Proteaser: | vol. 5 |
| Lipaser: | vol. 6 |

It is also contemplated that other enzyme activities may be included in the oral care compositions of the invention, either in addition to or instead of e.g. a dextranase and/or mutanase, for example proteases, such as papain, endoglucosidases, lipases, amylase and mixtures thereof.

The dextranase may be derived from a strain of the filamentous fungal genus Paecilomyces, in particular a strain of Paecilomyces lilacinum. Paecilomyces lilacium dextranase (available from Novo Nordisk A/S).

A mutanase suitable for use e.g. in combination with a dextranase in an oral care composition of the invention may be produced by filamentous fungi from the group including Trichoderma, in particular from a strain of Trichoderma harzianum, such as Trichoderma harzianum CBS 243.71, or Penicillium, in particular a strain of Penicillium funiculosum, such as Penicillium funiculosum NRRL 1768, or a strain of Penicillium lilacinum, such as Penicillium lilacinum NRRL 896, or a strain of Penicillium purpurogenum, such as the strain of Penicillium purpurogenum CBS 238.95, or a strain of the genus Pseudomonas, or a strain of Flavobacterium sp., or a strain of Bacillus circulanse or a strain of Aspergillus sp., or a strain of Streptomyces. The mutanase may also be derived from Penicillium purpurogenum.

An oral care composition of the invention may suitably have incorporated an amount of enzyme moiety, e.g. dextranase and/or mutanase, equivalent to an enzyme activity, calculated as enzyme activity units in the final oral care product, in the range of from is 0.001 KDU to 1000 KDU/ml, preferably from 0.01 KDU/ml to 500 KDU/ml, especially from 0.1 KDU/ml to 100 KDU/ml, and from 0.001 MU/ml to 1000 MU/ml, preferably from 0.01 MU/ml to 500 MU/ml, especially from 0.01 MU/ml to 100 MU/ml and from 0.01 MU/ml to 100 MU/ml, respectively.

For use in oral care compositions, the modified enzymes should show sufficient enzymatic activity at temperatures between 20° C. and 45° C., especially around 370C, as the temperature prevailing in the human mouth lies within this interval.

Oral Care Products

As explained above, the present invention also relates to oral care compositions and products comprising a modified enzyme as described herein. The oral care product may have any suitable physical form (i.e. paste, gel, liquid, powder, ointment, tablet, chewing gum, etc.). An “oral care product” can be defined as a product which can be used for maintaining or improving the oral hygiene in the mouth of humans and animals, by preventing formation of dental plaque, removing dental plaque, preventing and/or treating dental diseases, etc. Oral care products according to the invention also encompass products for cleaning dentures, artificial teeth and the like.

Examples of such oral care products include toothpastes, dental creams, gels or tooth powders, odontics, mouth washes, pre- or post brushing rinse formulations, chewing gum and lozenges.

Toothpastes and tooth gels typically include abrasive polishing materials, foaming agents, flavouring agents, humectants, binders, thickeners, sweetening agents, whitening/bleaching/stain removing agents, water, and optionally enzymes.

Mouth washes, including plaque removing liquids, typically comprise a water/alcohol solution, flavouring agents, humectants, sweeteners, foaming agents, colorants, and optionally enzymes.

Abrasive polishing material can also be incorporated into a dentifrice product of the invention. Suitable abrasive polishing material includes alumina and hydrates thereof, such as alpha alumina trihydrate, magnesium trisilicate, magnesium carbonate, kaolin, aluminosilicates, such as calcined aluminum silicate and aluminum silicate, calcium carbonate, zirconium silicate, and also powdered plastics, such as polyvinyl chloride, polyamides, polymethyl methacrylate, polystyrene, phenol-formaldehyde resins, melamine-formaldehyde resins, urea-formaldehyde resins, epoxy resins, powdered polyethylene, silica xerogels, hydrogels and aerogels and the like. Also suitable as abrasive agents are calcium pyrophosphate, water-insoluble alkali metaphosphates, dicalcium phosphate and/or its dihydrate, dicalcium orthophosphate, tricalcium phosphate, particulate hydroxylapatite and the like. It is also possible to employ mixtures of these substances.

Depending on the nature of the oral care product, the abrasive material may be present in an amount of from 0 to 70% by weight, preferably from 1% to 70%. For toothpastes, the abrasive material content typically lies in the range of from 10% to 70% by weight of the final toothpaste product.

Humectants are employed to prevent loss of water from e.g. toothpastes. Suitable humectants for use in oral care products according to the invention include the following compounds and mixtures thereof: glycerol, polyol, sorbitol, polyethylene glycols (PEG), propylene glycol, 1,3-propanediol, 1,4-butanediol, hydrogenated partially hydrolysed polysaccharides and the like. Humectants are in general present in an amount of from 0% to 80%, preferably 5 to 70% by weight in toothpaste.

Silica, starch, tragacanth gum, xanthan gum, extracts of Irish moss, alginates, pectin, cellulose derivatives, such as hydroxyethyl cellulose, sodium carboxymethyl cellulose and hydroxypropyl cellulose, polyacrylic acid and its salts, and polyvinylpyrrolidone are examples of suitable thickeners and binders that may be used to stabilise the dentifrice product. Thickeners may be present in toothpastes, creams and gels in an amount of from 0.1 to 20% by weight, and binders in an amount of from 0.01 to 10% by weight of the final product.

As a foaming agent, soaps as well as anionic, cationic, non-ionic, amphoteric and/or zwitterionic surfactants can be used. These may be present at levels of from 0% to 15%, preferably from 0.1 to 13%, more preferably from 0.25 to 10% by weight of the final product.

Surfactants are only suitable to the extent that they do not exert an inactivation effect on the modified enzymes. Surfactants include fatty alcohol sulphates, salts of sulphonated mono-glycerides or fatty acids having 10 to 20 carbon atoms, fatty acid-albumen condensation products, salts of fatty acids amides and taurines and/or salts of fatty acid esters of isethionic acid.

Suitable sweeteners include saccharin.

Flavours, such as spearmint, are usually present in low amounts, such as from 0.01% to about 5% by weight, especially from 0.1% to 5%.

Whitening/bleaching agents include H₂O₂ and may be added in amounts less that 5%, preferably from 0.25 to 4%, calculated on the basis of the weight of the final product.

Water is usually added in an amount sufficient to give the product, e.g. a toothpaste, a flowable form.

Furthermore, water-soluble anti-bacterial agents, such as chlorhexidine digluconate, hexetidine, alexidine, quaternary ammonium anti-bacterial compounds and water-soluble sources of certain metal ions such as zinc, copper, silver and tin (e.g. zinc, copper and stannous chloride, and silver nitrate) may also be included.

Also contemplated according to the invention is the addition of compounds which can be used as a fluoride source, dyes/colorants, preservatives, vitamins, pH-adjusting agents, anti-caries agents, desensitizing agents etc.

Enzymes provide several benefits when used for cleansing of the oral cavity. Proteases break down salivary proteins, which are adsorbed onto the tooth surface and form the pellicle, the first layer of resulting plaque. Proteases along with lipases destroy bacteria by lysing proteins and lipids which form the structural components of bacterial cell walls and membranes.

Dextranase breaks down the organic skeletal structure produced by bacteria that forms a matrix for bacterial adhesion. Proteases and amylases not only prevent plaque formation but also prevent the development of calculus by breaking up the carbohydrate-protein complex that binds calcium, preventing mineralization.

A toothpaste produced from an oral care composition of the invention (in weight % of the final toothpaste composition) may typically comprise the following ingredients:

| | |
|---|---|
| Abrasive material | 10 to 70% |
| Humectant | 0 to 80% |
| Thickener | 0.1 to 20% |
| Binder | 0.01 to 10% |
| Sweetener | 0.1% to 5% |
| Foaming agent | 0 to 15% |
| Whitener | 0 to 5% |
| Modified enzyme(s) | 0.0001% to 20% |

A mouth wash produced from an oral care composition of the invention (in weight % of the final mouth wash composition) may typically comprise the following ingredients:

| | |
|---|---|
| 0-20% | Humectant |
| 0-2% | Surfactant |
| 0-5% | Modified enzyme(s) |
| 0-20% | Ethanol |
| 0-2% | Other ingredients (e.g. flavour, sweetener, active |
| | ingredients such as fluorides). |
| 0-70% | Water |

The mouth wash may be in non-diluted form (i.e. to be diluted before use) or in ready-to-use form.

Use of an Oral Care Composition or Product

In the third aspect the invention relates to the use of the composition of the invention or an oral care product of the invention for preventing the formation of plaque or for removing dental plaque.

Using a product of the invention typically involves applying a safe and effective amount of said product to the oral cavity. These amounts (e.g. from 0.3 to about 2 grams), if it is a toothpaste or tooth gel, is kept in the mouth for a suitable period of time, e.g. from about 15 seconds to about 12 hours. It will be clear from the description above that even though a modified enzyme-containing oral care composition or product as such may only be kept in the mouth for a limited period of time, for example about 1-3 minutes for a toothpaste or mouthwash, the modified enzymes nevertheless become bound to tooth surfaces and therefore are able to exert an enzymatic action for an extended period of time.

Method of Manufacture

The oral care composition and products of the present invention can be made using methods which are common in the oral product area.

The invention will be further illustrated in the following non-limiting examples.

### MATERIALS AND METHODS

Enzymes:

Recombinant dextranase derived from Paecilomyces lilacinum (available from Novo Nordisk A/S).

Recombinant lipase derived from Thermomyces Lanuginosus (available from Novo Nordisk A/S).

Methods:

Preparation of Hydroxyapatite Disks

Hydroxyapatite disks are prepared by compressing 250 mg of hydroxyapatite in a disk die at about 5,900 kg (13,000 lbs) of pressure for 5 minutes. The disks are then sintered at 600° C. for 4 hours and finally hydrated with sterile de-ionised water.

Sterilization of Hydroxyapatite Disks

HAP disks are sterilised at 180° C. for two hours, hydrated with the sterilised de-ionised water and placed in a lid of Nunc tube (10 ml volume).

Determination of Dextranase Activity (KDU)

One Kilo Novo Dextranase Unit (1 KDU) is the amount of enzyme which breaks down dextran forming reducing sugar equivalent to 1 g maltose per hour in Novo Nordisk' method for determination of dextranase based on the following standard conditions:

| Substrate | Dextran 500 (Pharmacia) |
|---|---|
| Reaction time | 20 minutes |
| Temperature | 40° C. |
| pH | 5.4 |

A detailed description of Novo Nordisk's analytical method (AF 120) is available on request.

### EXAMPLES

### Example 1

Preparation of Modified Dextranase

Conjugation of dextranase with polyglutamic acid through carbodiimide-mediated coupling was performed according to standard procedures, see e.g. G. T Hermanson. Bioconjugate Techniques, Academic Press, 1996.

An enzyme stock solution of dextranase was diluted in 50 mM MES buffer containing 250 mM NaCl at pH 6.0. The final concentration of dextranase in the reaction mixture was 3.7 mg enzyme per ml.

The dextranase in the reaction mixture was activated by addition of 1-ethyl-3-(3-dimethylaminopropyl)carbodiimide (EDC; see Table 1) for one hour at ambient temperature.

After one hour the activated dextranase was purified by size-exclusion chromatography on a PD 10 column (Pharmacia). 50 mg of polyglutamic acid (Mᵣ 1000 D) (Sigma # p1818) was then added, and the coupling was allowed to proceed for 20 hours at room temperature.

The reaction was terminated and excess reagent was removed by dialysis for 65 hours against a sodium acetate buffer (10 mM at pH 5.5). The sodium acetate buffer was changed several times during this period.

The degree of reaction was followed by isoelectric focusing.

The conjugates produced in this manner were stored at 5° C.

TABLE 1
Preparation of dextranase conjugates

| Conjugate No. | [dextranase] (mg/ml) | [EDC] (mg/ml) | pI |
|---|---|---|---|
| 1 | 3.7 | 4.9 | 3.6-3.8 |
| 2 | 3.7 | 9.9 | 3.8-4.4 |

### Example 2

Hydroxylapatite Binding Test

500 microliters 10 mg/ml hydroxylapatite (HAP) in 50 mM Britton-Robinson buffer (at pH 4, 5, 6, 7, 8 and 9) was added to 500 microliters of a dextranase or dextranase conjugate solution (diluted in water to A₂₈₀=0.1). The resulting mixture was incubated at room temperature for 30 minutes while stirring. The samples were then centrifuged at 14,000 G for 4 minutes, and 500 microliters of the supernatant was diluted in 1.5 ml of water. The enzyme or modified enzyme concentration was then measured by fluorescence spectroscopy using a LS50 spectrometer from Perkin Elmer (excitation: 280 nm, emission: 340 nm). Controls without HAP addition were included, and the percentage of bound enzyme or modified enzyme was calculated relative to the control.

TABLE 2
Percent bound enzyme and modified enzyme

| Enzyme | pH 4 | pH 5 | pH 6 | pH 7 | pH 8 | pH 9 |
|---|---|---|---|---|---|---|
| Dextranase | 76 | 68 | 36 | 3 | 1 | 0 |
| Conjugate 1 | 81 | 76 | 64 | 39 | 19 | 3 |
| Conjugate 2 | 66 | 61 | 35 | 19 | 13 | 15 |

Conditions: 5 mg/ml HAP in 25 mM Britton-Robinson buffer. Enzyme or modified enzyme concentration: A₂₈₀ = 0.05.

### Example 3

Preparation of Modified Lipase

Conjugation of Lipolase with polyglutamic acid through carbodiimide-mediated coupling was performed according to standard procedures, see e.g. G. T Hermanson. Bioconjugate Techniques, Academic Press, 1996.

An enzyme stock solution of Lipolase was diluted in 50 mM MES buffer containing 250 mM NaCl at pH 6.0. The final concentration of Lipolase in the reaction mixture was 10 mg enzyme per ml.

Lipolase in the reaction mixture was activated by addition of 1-ethyl-3-(3-dimethylaminopropyl)carbodiimide (EDC; see Table 1) for one hour at ambient temperature.

After one hour the activated Lipolase was purified by size-exclusion chromatography on a PD 10 column (Pharmacia). 50 mg of polyglutamic acid (Mᵣ 2000-15000 D) (Sigma # P4636) was then added, and the coupling was allowed to proceed for 20 hours at room temperature.

The reaction was terminated and excess reagent was removed by dialysis for 16 hours against a sodium phosphate buffer (10 mM at pH 7).

The degree of reaction was followed by isoelectric focusing.

The conjugates produced in this manner were stored at 5° C.

TABLE 3
Preparation of Lipolase conjugates

| Conjugate No. | Lipolase (mg/ml) | EDC (mg/ml) | pI |
|---|---|---|---|
| 3 | 10 | 5 | 6.7 |
| 4 | 10 | 12 | 6.7 |

### Example 4

Hydroxylapatite Binding Test

500 microliters 10 mg/ml hydroxylapatit (HAP) in 50 mM Britton-Robinson buffer (at pH 4, 5, 6, 7, 8 and 9) was added 500 microliters enzyme (Lipolase, or EDC-poly-Glu modified Lipolase (conjugate no. 3 or no. 4 in example 3) diluted in water to A₂₈₀=0.1. The resulting mixture was incubated for 30 minutes at room temperature while stirring. Then the samples were centrifuged at 14,000 G for 4 minutes and 500 microliters of the supernatant was diluted into 1.5 ml water. The enzyme concentration was then measured by fluorescence spectroscopy using the LS50B spectrometer from Perkin Elmer (excitation: 280 nm, emission: 340 nm). Controls were included without HAP addition. Binding was calculated relative to the control.

TABLE 4
Percent bound enzyme and modified enzyme

| Enzyme | pH 4 | pH 5 | pH 6 | pH 7 | pH 8 |
|---|---|---|---|---|---|
| Lipolase | 47 | 33 | 12 | 11 | 4 |
| Conjugate No. 3 | 58 | 51 | 32 | 28 | 8 |

Conditions: 5 mg/ml HAP in 25 mM BR; Enzyme conc. A₂₈₀ = 0.05.

The results show an improved binding of the EDC-poly-Glu modified Lipolase (Conjugate No. 3) to hydroxylapatit in the entire pH-range.

TABLE 5
Percent bound enzyme and modified enzyme

| Enzyme | pH 5 | pH 7 |
|---|---|---|
| Lipolase | 38 | 11 |
| Conjugate No. 3 | 57 | 31 |
| Conjugate No. 4 | 68 | 61 |

Conditions: 5 mg/ml HAP in 25 mM BR; Enzyme conc. A₂₈₀ = 0.05

The results show a further improved binding of the EDC-poly-Glu modified Lipolase (Conjugate No. 4) to hydroxylapatit at both pH 5 and pH 6.

### Example 5

Synthesis of Oligomers of DL-2-Amino-3-phosphonoproprionic Acid and of DL-2-Amino-4-phosphonobutyric Acid

Oligomers of DL-2-Amino-3-phosphonopropionic acid and DL-2-Amino-4-phosphonobutyric acid were synthesized from the corresponding monomers (CAS: 20263-06-3, 20263-07-4) (SIGMA) using a three fold excess of carbonyldiimidazole (CDI) as a condensing agent (K. W. Ehler and L. E. Orgel, Biochim. Biophys. Acta, 434 (1976) 233-243.

84.5 mg DL-2-amino-3-phosphonopropionic acid was dissolved in 5 ml imidazole buffer (pH=7, 1M) at room temperature. The mixture was cooled to 0° C. and 260 mg imidazole was added in small potions. The mixture was slowly allowed to reach room temperature and stirring was continued for three days.

The synthesis of oligomers of DL-2-amino-4-phosphonobutyric acid was done in the same way as described above for the synthesis of oligomers of DL-2-amino-3-phosphonopropionic acid using 105 mg DL-2-amino-4-phosphonobutyric acid and 280 mg imidazole.

### Example 6

Preparation of Modified Lipase

Conjugation of Lipolase with the oligomer of DL-2-amino-3-phosphonopropionic acid through carbodiimide-mediated coupling was performed according to standard processes, see e.g. G. T Hermanson. Bioconjugate Techniques, Academic Press, 1996.

An enzyme stock solution of Lipolase was diluted in 50 mM MES buffer containing 250 mM NaCl at pH 6.0. The final concentration of Lipolase in the reaction mixture was 9.2 mg enzyme per ml.

The Lipolase in the reaction mixture was activated by addition of 1-ethyl-3-(3-d ethylaminopropyl)carbodiimide (EDC; see Table 1) for two hours at ambient temperature.

After two hours the activated Lipolase was purified by size-exclusion chromatography on a PD 10 column (Pharmacia). 2.5 ml of the oligomer of DL-2-amino-3-phosphonopropionic acid per ml of EDC-activated-Lipolase was added, and the coupling was allowed to proceed for 16 hours at ambient temperature.

The reaction was terminated and excess reagent was removed by dialysis for 16 hours at 5° C. against a sodium phosphate buffer (10 mM at pH 6).

The degree of reaction was followed by isoelectric focusing. The conjugate no. 5 produced in this manner was stored at 5° C.

Conjugate no. 6 was produced essentially through a similar route though by addition of 2.5 ml of the oligomer of DL-2-amino-3-phosphonobutyricic acid per ml of EDC-activated-Lipolase. The coupling was allowed to proceed for 16 hour at ambient temperature followed by dialysis as described above and storage of conjugate no. 6 at 5C.

TABLE 6
Preparation of lipolase conjugates

| Conjugate No. | Lipolase (mg/ml) | EDC (mg/ml) | pI |
|---|---|---|---|
| 5 | 9.2 | 10 | 9.9 |
| 6 | 9.2 | 10 | 9.6 |

### Example 7

Hydroxylapatite Binding Test

500 microliters 10 mg/ml hydroxylapatit (HAP) in 50 mM Britton-Robinson buffer (at pH 4, 5, 6, 7 and 8) was added 500 microliters enzyme (Lipolase, or phosphono derivate modified Lipolase (conjugate no. 5 or no. 6 in example 6) diluted in water to A₂₈₀=0.1. The resulting mixture was incubated for 30 minutes at room temperature while stirring. Then the samples were centrifuged at 14,000 G for 4 minutes and 500 microliters of the supernatant was diluted into 1.5 ml water. The enzyme concentration was then measured by fluorescence spectroscopy using the LS50B spectrometer from Perkin Elmer (excitation: 280 nm, emission: 340 nm). Controls were included without HAP addition. Binding was calculated relative to the control.

TABLE 7
Percent bound enzyme and modified enzyme

| Enzyme | pH 4 | pH 5 | pH 6 | pH 7 | pH 8 |
|---|---|---|---|---|---|
| Lipolase | 47 | 33 | 12 | 11 | 4 |
| Conjugate No. 5 | 61 | 69 | 69 | 62 | 48 |
| Conjugate No. 6 | 77 | 83 | 83 | 73 | 56 |

Conditions: 5 mg/ml HAP in 25 mM BR; Enzyme conc. A₂₈₀ = 0.05.

The results shows a significant better binding of the Lipolase conjugates to HAP compared to the Lipolase control in the entire pH range.

1. A modified enzyme comprising an enzyme and at least one polyanionic domain, wherein the enzyme comprises or is covalently attached to each said polyanionic domain.

2. The modified enzyme of claim 1, wherein said polyanionic domain is attached to the C-terminal carboxylate group or to the N-terminal amino group of the enzyme, or wherein the polyanionic domain is incorporated into the amino acid sequence of the enzyme.

3. The modified enzyme of claim 2, said modified enzyme being produced by means of recombinant DNA-technology.

4. The modified enzyme of claim 1 or 2, wherein said polyanionic domain is covalently attached to a carboxylate group, an amino group, a thiol group, a hydroxyl group, and/or an aldehyde group of the enzyme.

5. The modified enzyme of any of the preceding claims, wherein said polyanionic domain is covalently attached to a carboxylate group and/or an amino group of the enzyme.

6. The modified enzyme of claim 1, 4 or 5, wherein said polyanionic domain is selected from the group consisting of: compounds of the general formula I 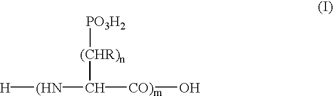
wherein n is an integer in the range of from 1 to 15, m is an integer in the range of from 1 to 50, and each R is independently selected from the group consisting of hydrogen, C₁₋₆-alkyl, C₂₋₆-alkenyl, hydroxy, amino, and halogen such as fluoro, chloro, iodo, and bromo; polycarboxylic acids; and aminated polycarboxylic acids.

7. The modified enzyme of any of claims 1-5, wherein said polyanionic domain is selected from the group consisting of peptides comprising from 1 to 150 amino acid residues, said peptides having a net negative charge at pH 7, preferably peptides comprising from 1 to 50 amino acid residues.

8. The modified enzyme of claim 7, wherein said polyanionic domain is selected from polyglutamic acid and/or polyaspartic acid, or wherein said polyanionic domain comprises a total of from 3 to 10 glutamic acid and/or aspartic acid residues.

9. The modified enzyme of any of the preceding claims, wherein the polyanionic domain is covalently attached to the enzyme by means of at least one C—N bond, the carbon atom originating from the enzyme and the nitrogen atom originating from the polyanionic domain.

10. The modified enzyme of any of claims 1-9, wherein the polyanionic domain is covalently attached to the enzyme by means of at least one C—N bond, the carbon atom originating from the polyanionic domain and the nitrogen atom originating from the enzyme.

11. The modified enzyme of claim 9 or 10, wherein the covalent bond between said enzyme and said polyanionic domain is a peptide bond.

12. The modified enzyme of any of the preceding claims, wherein said enzyme is selected from the group consisting of oxidoreductases, proteases, lipases, glucanases, esterases, deaminases, ureases and polysaccharide hydrolases.

13. The modified enzyme of claim 12, wherein the enzyme is a glucanase, in particular a dextranase and/or a mutanase.

14. The modified enzyme of any of the preceding claims, wherein the catalytic activity of said modified enzyme is at least 1% of the catalytic activity of the free enzyme, preferably at least 2%, such as at least 5%, e.g. at least 10%, more preferably at least 20%, such as at least 30%, e.g. at least 40%, still more preferably at least 50%, such as at least 60%, e.g. at least 70%, even more preferably at least 80%, such as at least 90%, e.g. at least 95%, most preferably the modified enzyme is substantially identical to the catalytic activity of the free enzyme, as determined according to “Methods of Enzymatic Analysis”, 3rd. Edition, vol. 1-10, 1984, Verlag Chemie, Weinheim.

15. The modified enzyme of any of the proceeding claims, wherein said modified enzyme is capable of binding to hydroxylapatite (HAP), fluoroapatite, calcium phosphate, teeth or bone.

16. The modified enzyme of claim 15, wherein the amount of modified enzyme which binds to HAP is at least 5% as defined in the “Hydroxylapatite binding test” at pH 7, preferably at least 10%, such as at least 20%, e.g. at least 30%, more preferably at least 40%, such as at least 50%, e.g. at least 60%, still more preferably at least 70%, such as at least 80%, e.g. at least 90%, most preferably at least 95%, such as at least 99%.

17. An oral care composition comprising at least one modified enzyme as defined in any of claims 1-16.

18. Use of an oral care composition or oral care product, said oral care composition or oral care product comprising at least one modified enzyme as defined in any of claims 1-16, for the prevention or treatment of a dental disease, in particular for preventing the formation of dental plaque or removing dental plaque.

19. An oxidoreductase modified by covalent bonding to one or more polyglutamate and/or polyaspartate anions.

20. The enzyme of claim 19, which is a laccase.

21. The enzyme of claim 20, wherein the laccase is derived from a strain of Myceliophthora thermophila.

22. The enzyme of claim 19, which is a glucose oxidase.

23. The enzyme of claim 22, wherein the glucose oxidase is derived from Aspergillus niger.

24. The enzyme of claim 19, wherein the one or more polyglutamate and/or polyaspartate anions are attached to the C-terminal carboxylate group or to the N-terminal amino group of the enzyme, or wherein the one or more polyglutamate and/or polyaspartate anions incorporated into the amino acid sequence of the enzyme.

25. The enzyme of claim 19, wherein the one or more polyglutamate and/or polyaspartate anions are covalently bound to a carboxylate group, an amino group, a thiol group, a hydroxyl group, and/or an aldehyde group of the enzyme.

26. The enzyme of claim 19, wherein the one or more polyglutamate and/or polyaspartate anions are covalently bound to a carboxylate group and/or an amino group of the enzyme.

27. The enzyme of claim 19, wherein the one or more polyglutamate and/or polyaspartate anions are selected from the group consisting of Glu-Glu, (Glu)₃, (Glu)₄, (Glu)₅, (Glu)₆, (Glu)₇, (Glu)₈, (Glu)₉, (Glu)₁₀, Asp-Asp, (Asp)₃, (Asp)₄, (Asp)₅, (Asp)6, (Asp)₇, (Asp)₈, (Asp)₉, (Asp)₁₀, Glu-Asp, (Glu-Asp)₂, (Glu-Asp)₃, (Glu-Asp)₄, (Glu-Asp)₅, Asp-Glu, (Asp-Glu)₂, (Asp-Glu)₃, (Asp-Glu)₄, (Asp-Glu)₅, Xaa-Glu, (Xaa-Glu)₂, (Xaa-Glu)₃, (Xaa-Glu)₄, (Xaa-Glu)₅, (Xaa-Glu)₆, (Xaa-Glu)₇, (Xaa-Glu)₈, (Xaa-Glu)₉, (Xaa-Glu)₁₀, Glu-Xaa, (Glu-Xaa)₂, (Glu-Xaa)₃, (Glu-Xaa)₄, (Glu-Xaa)₅, (Glu-Xaa) 6, (Glu-Xaa)₇, (Glu-Xaa)₈, (Glu-Xaa)₉, (Glu-Xaa)₁₀, Xaa-Asp, (Xaa-Asp)₂, (Xaa-Asp)₃, (Xaa-Asp)₄, (Xaa-Asp)₅, (Xaa-Asp)₆, (Xaa-Asp)₇, (Xaa-Asp)₈, (Xaa-Asp)₉, (Xaa-Asp)₁₀, Asp-Xaa, (Asp-Xaa) 2 (Asp-Xaa)₃, (Asp-Xaa)₄, (Asp-Xaa)₅, (Asp-Xaa)₆, (Asp-Xaa)₇, (Asp-Xaa)₈, (Asp-Xaa)₉, (Asp-Xaa)₁₀, and Xaaₐ-(Glu or Asp)_{b}-Xaa_{c}-(Glu or Asp)_{d}-Xaaₑ, wherein Xaa is alanine, valine, leucine, isoleucine, methionine, phenylalanine, tryptophan, proline, glycine, serine, threonine, cysteine, tyrosine, aspargine or glutamine, and a, b, c, d and e are integers in the range of from 0 to 25.

28. A composition comprising (a) an enzyme of claim 19 and (b) one or more oral agents selected from the group consisting of abrasive polishing materials, foaming agents, flavoring agents, humectants, binders, thickeners, sweetening agents, whitening/bleaching/stain removing agents, plaque removing liquids, colorants and surfactants.

29. The oral composition of claim 28, which is a toothpaste, dental cream, gel or powder, odontic, mouthwash, pre- or post-brushing rinse formulation, chewing gum or lozenge.

30. The oral composition of claim 28, wherein the catalytic activity of the modified enzyme is at least 1% of the catalytic activity of the unmodified enzyme.

31. A method of preventing or treating a dental disease, comprising applying an oral composition of claim 28 to teeth.

32. A method of bleaching teeth, comprising applying an oral composition of claim 28 to the teeth.

33. A method of reducing halitosis, comprising applying an oral composition of claim 28 to teeth.
